(19) United States
(12) Patent Application Publication
(10) Pub. No.: US 2007/0078445 A1
(43) Pub. Date: Apr. 5, 2007

(54) SYNCHRONIZATION APPARATUS AND METHOD FOR IONTOPHORESIS DEVICE TO DELIVER ACTIVE AGENTS TO BIOLOGICAL INTERFACES

(76) Inventor: Curt Malloy, Sammamish, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/533,260

(22) Filed: Sep. 19, 2006

Related U.S. Application Data
(60) Provisional application No. 60/722,088, filed on Sep. 30, 2005.

(51) Int. Cl. A61K 9/22; A61M 37/00

(52) U.S. Cl. 604/890.1

(57) ABSTRACT

Active agent delivery devices, for example iontophoresis devices, adjust active agent delivery based at least in part on parameters and/or other performance information received from other active agent delivery devices. The delivery devices may monitor parameters (e.g., current, voltage, time, impedance, active agent identity) and wireless transmit signals indicative of performance information to other delivery devices. The delivery devices may operate sequentially, or simultaneously. The delivery devices may form a repeater system. The devices may monitor for combinations of active agents with likely adverse interactions, or for active agents for which the subject may have a known or suspected adverse reaction.

### CROSS-REFERENCE TO RELATED APPLICATION

This application claims the benefit under 35 U.S.C. § 119(e) of U.S. Provisional Patent Application No. 60/722,088, filed Sep. 30, 2005.

### BACKGROUND OF THE INVENTION

1. Field of the Invention

This disclosure generally relates to the field of iontophoresis, and more particularly to the delivery of active agents such as therapeutic agents or drugs to a biological interface under the influence of electromotive force.

2. Description of the Related Art

Iontophoresis employs an electromotive force to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate voltage polarity based on the polarity of the active agent. lontophoresis may be advantageously used to enhance or control the delivery rate of the active agent. As discussed in U.S. Pat. No. 5,395,310, the active agent may be stored in a reservoir such as a cavity. Alternatively, the active agent may be stored in a reservoir such as a porous structure or a gel. Also as discussed in U.S. Pat. No. 5,395,310, an ion exchange membrane may be positioned to serve as a polarity selective barrier between the active agent reservoir and the biological interface.

It may be desirable to provide a particular treatment regime over an extended period of time, and/or involving two or more distinct active agents that must be delivered sequentially, or that must be delivered simultaneously to two distinctly different areas or that cannot be mixed together. While it is possible to use two or more iontophoresis devices, simultaneously and/or sequentially, it may be difficult to achieve a desired delivery profile. In particular, it may be difficult to accommodate for the interaction between the delivery regimes of the different iontophoresis devices. Such may have adverse consequences, for example delivering an overdose of active agent, or delivering two different active agents the interaction of which produces an undesired reaction.

Commercial acceptance of iontophoresis devices is dependent on a variety of factors, such as cost to manufacture, shelf life or stability during storage, efficiency and/or timeliness of active agent delivery, biological capability, disposal issues and/or ease of use and ability to deliver a desired profile over an extended period of time. An iontophoresis device that addresses one or more of these factors is desirable.

### BRIEF SUMMARY OF THE INVENTION

In at least one embodiment, an active agent device operable to deliver active agent to a biological entity includes an active agent reservoir to hold a quantity of the active agent, a power source operable to supply power to actively transfer at least some of the active agent from the active agent delivery device to the biological interface, a monitoring circuit operable to monitor at least one parameter indicative of a transfer of active agent from the device, at least a first antenna, and a transmitter coupled to the at least one antenna to transmit a signal indicative of at least one of the monitored parameters.

In another embodiment, an active agent delivery system operable to control delivery of an active agent to a biological entity includes a first active agent delivery device including an active agent reservoir to hold a quantity of the active agent, a control circuit operable to control and monitor at least one aspect of a delivery of the active agent from the first active agent delivery device, at least one antenna operable to transmit a signal indicative of at least one of the monitored aspects, and at least a second active agent delivery device including an active agent reservoir to hold a quantity of the active agent, a control circuit operable to control and monitor at least one aspect of a delivery of the active agent from the second active agent delivery device, at least one antenna operable to receive the signal indicative of at least one of the monitored aspects of the first active agent delivery device.

In yet another embodiment, a method of operating at least a first and a second active agent delivery device to deliver an active agent to a biological entity includes delivering a quantity of an active agent from the first active agent delivery device, monitoring at least one aspect of the delivery of the active agent from the first active agent delivery device, transmitting a signal to the second active agent delivery device at least indicative of the at least one monitored aspect of the delivery of the active agent from the first active agent delivery device, and delivering a quantity of an active agent from the second active agent delivery device, based in part on information in the signal received from the first active agent delivery device.

In still yet another embodiment, a method of operating at least a first and a second active agent delivery device to deliver an active agent to a biological entity includes delivering a quantity of an active agent from the first active agent delivery device, monitoring at least one aspect of the delivery of the active agent from the first active agent delivery device, transmitting a signal to the second active agent delivery device at least indicative of the at least one monitored aspect of the delivery of the active agent from the first active agent delivery device, and delivering a quantity of an active agent from the second active agent delivery device, based in part on information in the signal received from the first active agent delivery device.

In still yet another embodiment, a method of operating a plurality of active agent delivery devices to deliver active agents to a biological entity includes delivering a quantity of a first active agent from a first one of the active agent delivery devices, monitoring at least one aspect of the delivery of the first active agent from the first active agent delivery device, transmitting a signal to at least a second one of the active agent delivery devices indicative of the at least one monitored aspect of the delivery of the first active agent from the first active agent delivery device, delivering a quantity of a second active agent from the second active agent delivery device, based in part the at least one monitored aspect of delivery of the first active agent from the first active agent delivery device, monitoring at least one aspect of the delivery of the second active agent from the second active agent delivery device, transmitting a signal to at least a third one of the active agent delivery devices indicative of the at least one monitored aspect of the delivery of the second active agent from the second active agent delivery device, and delivering a quantity of a third active agent from the third active agent delivery device, based in part on the at least one monitored aspect of delivery of at least one of the first and the second active agents from the first and the second active agent delivery devices.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a block diagram of an active agent delivery device in the form of an iontophoresis device comprising active and counter electrode assemblies, a controller, radio transmitter and antenna, regulator and power source, according to one illustrated embodiment.

FIG. 2 is a top plan view of an active agent delivery device that positions an antenna active radiating element in the form of a dipole antenna over an electrode element to form an antenna system, according to one illustrated embodiment.

FIG. 3 is a cross-sectional view of the active agent delivery device of FIG. 2.

FIG. 4 is a top plan view of an active agent delivery device including a ground plane forming an antenna system with an antenna active radiating element in the form of a coil antenna, according to another illustrated embodiment.

FIG. 5 is a schematic diagram showing a first active agent delivery device positioned on a biological interface, exchanging information with a second active agent delivery device, according to one illustrated embodiment.

FIG. 6 is a schematic diagram showing the first active agent delivery device removed from the biological interface, the second active agent delivery device positioned on the biological interface, exchanging information with a third active agent delivery device, according to one illustrated embodiment.

FIG. 7 is a schematic diagram showing a first active agent delivery device positioned on a biological interface, exchanging information with a second and a third active agent delivery device, according to one illustrated embodiment.

FIG. 8 is a schematic diagram showing a three active agent delivery devices positioned on a biological interface and exchanging information therebetween, according to one illustrated embodiment.

FIG. 9 is a high level flow diagram of a method of operating an active agent delivery device to monitor and report parameters and/or performance information, according to one illustrated embodiment.

FIG. 10 is a high level flow diagram of a method of operating an active agent delivery device to receive parameters and/or performance information and modify active agent delivery in response thereto, according to one illustrated embodiment.

FIG. 11 is a low level flow diagram of a method of determining whether to terminate operation according to one illustrated embodiment, the method useful in the methods of FIGS. 9 and 10.

FIG. 12 is a low level flow diagram of a method of determining whether to report parameters and/or performance information according to one illustrated embodiment, the method useful in the method of FIG. 9.

FIG. 13 is a low level flow diagram of a method of monitoring parameters and/or performance information according to one illustrated embodiment, the method useful in the method of FIG. 9.

FIG. 14 is a low level flow diagram of a method of monitoring parameters and/or performance by monitoring a current through a reservoir, membrane or other structure of the active agent delivery device according to one illustrated embodiment, the method useful in the method of FIG. 9.

FIG. 15 is a low level flow diagram of a method of monitoring parameters and/or performance by monitoring a voltage across a reservoir, membrane or other structure of the active agent delivery device according to one illustrated embodiment, the method useful in the method of FIG. 9.

FIG. 16 is a low level flow diagram of a method of monitoring parameters and/or performance information by comparing an identity of first and second active agents for adverse interactions, according to one illustrated embodiment, the method useful in the method of FIG. 9.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane which substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein and in the claims, the term bipolar membrane means a membrane that is selective to two different charges or polarities. Unless specified otherwise, a bipolar membrane may take the form of a unitary membrane structure or multiple membrane structure. The unitary membrane structure may having a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

A used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

The headings provided herein are for convenience only and do not interpret the scope or meaning of the embodiments.

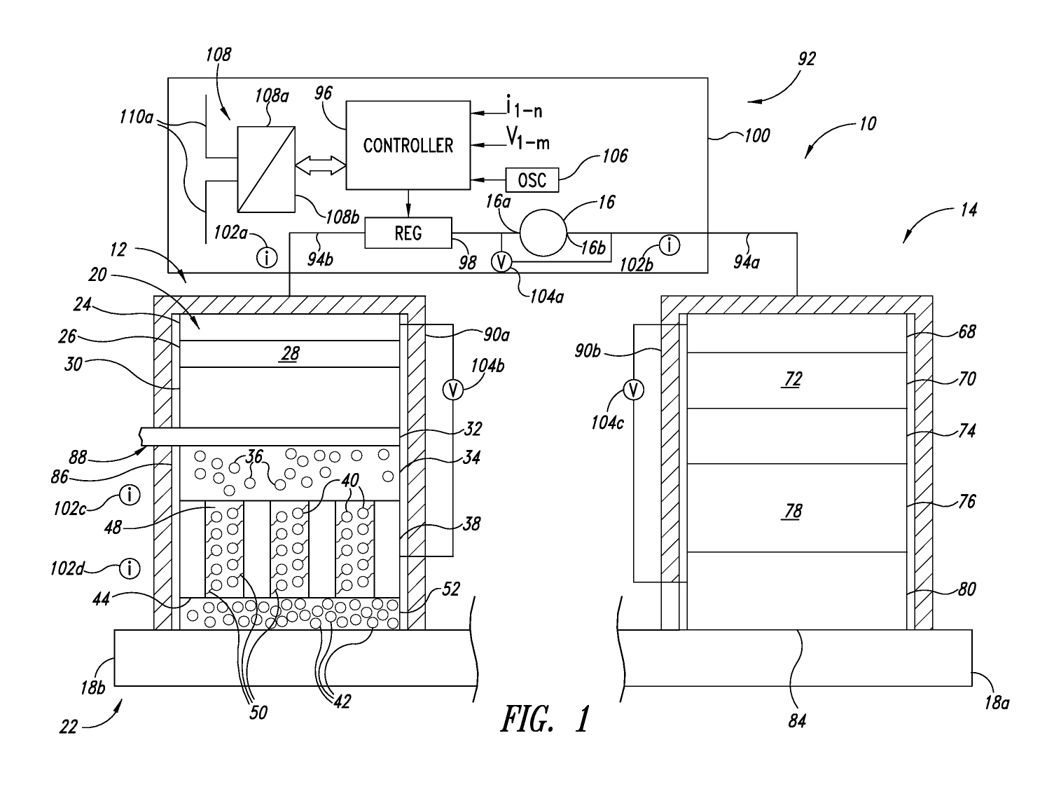

FIG. 1 shows an active agent delivery device in the form of an iontophoresis device 10, comprising: an active electrode assembly 12 positioned on or proximate a first portion 18b of a biological interface 18, and counter assembly 14 positioned proximate a second portion 18a of the biological interface 18, each electrode assembly 12, 14 electrically coupled to a power source 16 and operable to supply at least one active agent to the second portion 18b of the biological interface 18 via iontophoresis, according to one illustrated embodiment. As noted above, the biological interface 18 may take a variety of forms, for example, a portion of skin, mucous membrane, gum, tooth or other tissue.

In the illustrated embodiment, the active electrode assembly 12 comprises, from an interior 20 to an exterior 22 of the active electrode assembly 12, an active electrode element 24, an electrolyte reservoir 26 storing an electrolyte 28, an inner ion selective membrane 30, an optional inner sealing liner 32, an inner active agent reservoir 34 storing active agent 36, an outermost ion selective membrane 38 that caches additional active agent 40, and further active agent 42 carried by an outer surface 44 of the outermost ion selective membrane 38. Each of the above elements or structures will be discussed in detail below.

The active electrode element 24 is coupled to a first pole 16a of the power source 16 and positioned in the active electrode assembly 12 to apply an electromotive force or current to transport active agent 36, 40, 42 via various other components of the active electrode assembly 12. The active electrode element 24 may take a variety of forms. For example, the active electrode element 24 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 24. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

The electrolyte 28 may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. As discussed further below, in some embodiments the electrolyte 28 may provide or donate ions to substitute for the active agent, for example substituting for the active agent 40 cached in the outermost ion selective membrane 39. Such may facilitate transfer of the active agent 40 to the biological interface 18, for example, increasing and/or stabilizing delivery rates. A suitable electrolyte may take the form of a solution of 0.5M disodium fumarate: 0.5M Poly acrylic acid (5:1).

The inner ion selective membrane 30 is generally positioned to separate the electrolyte 28 and the inner active agent reservoir 34. The inner ion selective membrane 30 may take the form of a charge selective membrane. For example, where the active agent 36, 40, 42 comprises a cationic active agent, the inner ion selective membrane 38 may take the form of an anion exchange membrane, selective to substantially pass anions and substantially block cations. Also, for example, where the active agent 36, 40, 42 comprises an anionic active agent, the inner ion selective membrane 38 may take the form of an cationic exchange membrane, selective to substantially pass cations and substantially block anions. The inner ion selective membrane 38 may advantageously prevent transfer of undesirable elements or compounds between the electrolyte 28 and the active agents 26, 40, 42. For example, the inner ion selective membrane 38 may prevent or inhibit the transfer of hydrogen (H⁺) or sodium (Na⁺) ions from the electrolyte 72, which may increase the transfer rate and/or biological compatibility of the iontophoresis device 10.

The optional inner sealing liner 32 separates the active agent 36, 40, 42 from the electrolyte 28 and is selectively removable via slot or opening 88. The inner sealing liner 32 may advantageously prevent migration or diffusion between the active agent 36, 40, 42 and the electrolyte 28, for example, during storage.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36, and in some embodiments may even be the active agent 36 itself, for example, where the active agent 36 is in a gel, semi-solid or solid form. For example, the inner active agent reservoir 34 may take the form of a pouch or other receptacle, a membrane with pores, cavities or interstices, particularly where the active agent 36 is a liquid. The inner active agent reservoir 34 may advantageously allow larger doses of the active agent 36 to be loaded in the active electrode assembly 12.

The outermost ion selective membrane 38 is positioned generally opposed across the active electrode assembly 12 from the active electrode element 24. The outermost membrane 38 may, as in the embodiment illustrated in FIG. 1, take the form of an ion exchange membrane, pores 48 (only one called out in FIG. 1 for sake of clarity of illustration) of the ion selective membrane 38 including ion exchange material or groups 50 (only three called out in FIG. 1 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., strontium, lidocaine), the outermost ion selective membrane 38 may take the form of a cation exchange membrane. Alternatively, where the active agent 36, 40, 42 is an anion (e.g., fluoride), the outermost ion selective membrane 38 may take the form of an anion exchange membrane.

The outermost ion selective membrane 38 may advantageously cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing a removably releasable outer release liner 46 (FIG. 3) to retain the active agent 40 until the outer release liner 46 is removed prior to use.

The outermost ion selective membrane 38 may be preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

An interface coupling medium (not shown) may be employed between the electrode assembly and the biological interface 18. The interface coupling medium may, for example, take the form of an adhesive and/or gel. The gel may, for example, take the form of a hydrating gel.

The power source 16 may take the form of one or more chemical battery cells, super- or ultra-capacitors, or fuel cells. The power source 16 may, for example, provide a voltage of 12.8V DC, with tolerance of 0.8V DC, and a current of 0.3 mA. The power source 16 may be selectively electrically coupled to the active and counter electrode assemblies 12a, 14 via a control circuit 92 (discussed below), for example, via carbon fiber ribbons 94a, 94b. The iontophoresis device 10 may include a controller 96 and a regulating circuit 98 (discussed below) formed from discrete and/or integrated circuit elements to control and/or monitor operation, and/or regulate the voltage, current and/or power delivered to the electrode assemblies 12a, 14. For example, the iontophoresis device 10a may include a diode to provide a constant current to the electrode elements 20, 40.

As suggested above, the active agent 24 may take the form of a cationic or an anionic drug or other therapeutic agent. Consequently, the poles or terminals of the power source 16 may be reversed. Likewise, the selectivity of the outermost ion selective membranes 22, 42 and inner ion selective membranes 34, 54 may be reversed.

The control circuit 92 includes the controller 96 and regulating circuit 98, which may be mounted or carried by a circuit board, such as flexible circuit board 100. The flexible circuit board 100 may comprises one or more insulative layers, and may optionally comprise one or more conductive layers interlaced with the insulative layers. The circuit board 100 may form one or more vias (best illustrated in FIG. 3), to make electrically couplings between the surfaces of the circuit board and/or between various ones of the conductive layers.

The control circuit 92 may also include one or more current sensors 102a-102d (collectively 102), positioned and configured to sense or measure current through one or more reservoirs, membranes or other structures. The control circuit 92 may also include one or more voltage sensors 104a-104c (collectively 104), positioned and configured to sense or measure voltage across one or more reservoirs, membranes or other structures. The current and voltage sensors 102, 104 provide signals indicative of the current i₁-iₙ, and signals indicative of the voltage v₁-vₘ, respectively, to the controller 96.

The control circuit 92 may also include an off-chip oscillator 106 that provides a frequency signal to the controller 96 to form a clock signal. Alternatively, the controller 92 may employ an on-chip oscillator.

The controller 92 may employ the signals indicative of the current i₁-iₙ, and signals indicative of the voltage v₁₋ᵥₘ, as well as the frequency signals to analyze operation of the device, and to produce additional performance information, as discussed in more detail below.

The device 10 also includes a transmitter 108a and/or receiver 108b which may be formed as a transceiver 108, which may be coupled to one or more active radiating antenna elements, for example dipole antenna 110a. The controller 92 is communicatively coupled to receive and/or provide information from and/or to the transceiver 108. Thus, the controller 92 may cause the transmitter 108a to transmit parameter and/or performance information from the iontophoresis device 10. Likewise, the controller 92 may receive parameter and/or performance information from another iontophoresis device 10 via the receiver 108b.

The controller 96 may use the parameter and/or other performance information that it generates, as well as parameters and/or other performance information received from other active agent delivery devices to modify the active agent delivery regime. For example, the controller 96 may determine a new or updated active agent delivery regime based on the parameters and/or other performance information, and provide appropriate control signals to the regulating circuit to implement the new or revised regime. The regulating circuit 98 may take the form a voltage control regulator and/or current control regulator, that controls the delivery of active agent by controlling voltage applied across, or current applied to, the electrode elements 24, 68.

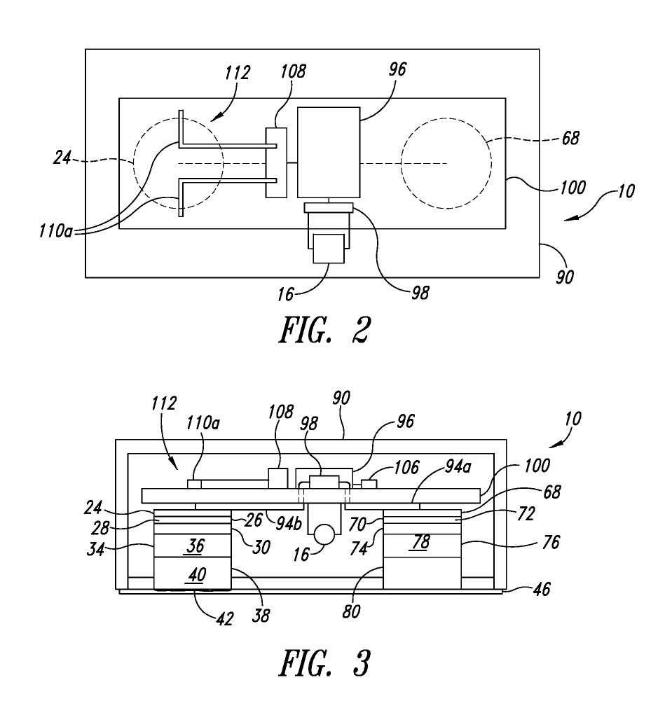

FIGS. 2 and 3 shows an active agent delivery device in the form of an iontophoresis device 10. Many structures and operations are similar to that of the embodiment of FIG. 1, and are identified with common reference numerals. Only significant differences in structure and/or operation will be discussed, in the interest of brevity and clarity.

The illustrated embodiment advantageously locates the active radiating antenna element (e.g., dipole antenna 110) over one of the electrode elements, for example the active electrode element 24. This positioning causes the active electrode element 24 to function as a passive radiating antenna element. The active radiating antenna element (e.g., dipole antenna 110) and passive radiating antenna element (e.g., active electrode element) form an antenna system 112. The circuit board 100 may optionally provide a dielectric interface between the active and passive radiating antenna elements. The antenna system 112 may have improved range and higher directionality than the dipole antenna alone. Higher directionality may reduce interference from other sources of radio signals, and/or reduce the possibility of eavesdropping or receiving intentionally or unintentionally incorrect information. Increased range may advantageously facilitate operation or use amongst a plurality of devices, and may advantageously reduce power consumption.

In particular, the dipole antenna 110 is spaced distally from the active electrode element 24 with respect to a portion of the device that will contact or be proximate the biological interface 18. This advantageously provide directionality in a direction away from the biological interface 18, reducing interference by the biological interface 18 and thus increasing range, and/or reducing any absorption of radio signals by the biological interface 18.

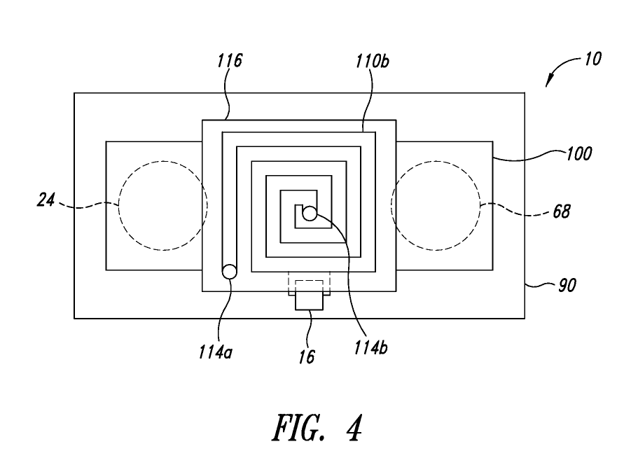

FIG. 4 shows an active agent delivery device in the form of an iontophoresis device 10. Many structures and operations are similar to that of the embodiments of FIGS. 1, 2 and 3, and are identified with common reference numerals. Only significant differences in structure and/or operation will be discussed, in the interest of brevity and clarity.

In particular, FIG. 4 shows the active radiating antenna element formed as a coil antenna 110b, electrically coupled to the transceiver 108 by vias 114a, 114b. Instead of positioning the coil antenna 110b over one of the electrode elements 24, 68, the embodiment employs a distinct passive radiating antenna element 116. Such may, for example, take the form of a ground plane formed on or in a portion of the circuit board 100, or a structure distinct from the circuit board 100.

Other embodiments may employ additional passive radiating antenna elements. Still other embodiments many omit all passive radiating antenna elements, depending on the range and/or directionality requirements of the particular application.

FIG. 5 shows a first active agent delivery device 10a in the form of a iontophoresis patch applied to a biological interface 18, to deliver active agent thereto. The first active agent delivery device 10a wireless communicates with a second active agent delivery device 10b, in the form of an iontophoresis patch that is not attached to the biological interface 18. The second active agent delivery device 10b may have recently been removed from the biological interface 18, and may be providing parameters and/or other performance information to the first active agent delivery device 10a. The first active agent delivery device 10a may use the received parameters and/or other performance information to control a delivery of the active agent to the biological interface 18.

Alternatively, the second active agent delivery device 10b may be waiting to be applied to the biological interface 18 either before, or after, removal of the first active agent delivery device 10a. Thus, the second active agent delivery device 10b may be receiving parameters or performance information from the first active agent delivery device 10a in preparation to deliver active agent from the second active agent delivery device 10b once placed in use.

In particular, FIG. 6 shows the first active agent delivery device 10a removed from the biological interface 18, and the second active agent delivery device 10b applied to the biological interface 18 to deliver active agent thereto. The second active agent delivery device 10b wirelessly communications parameters or other performance information to a third active agent delivery device 10c, in preparation for the third active agent delivery device 10c being placed in use. The arrangement illustrated in FIG. 6 may follow, that illustrated in FIG. 5, where the active agent delivery devices 10a-10c are employed sequentially.

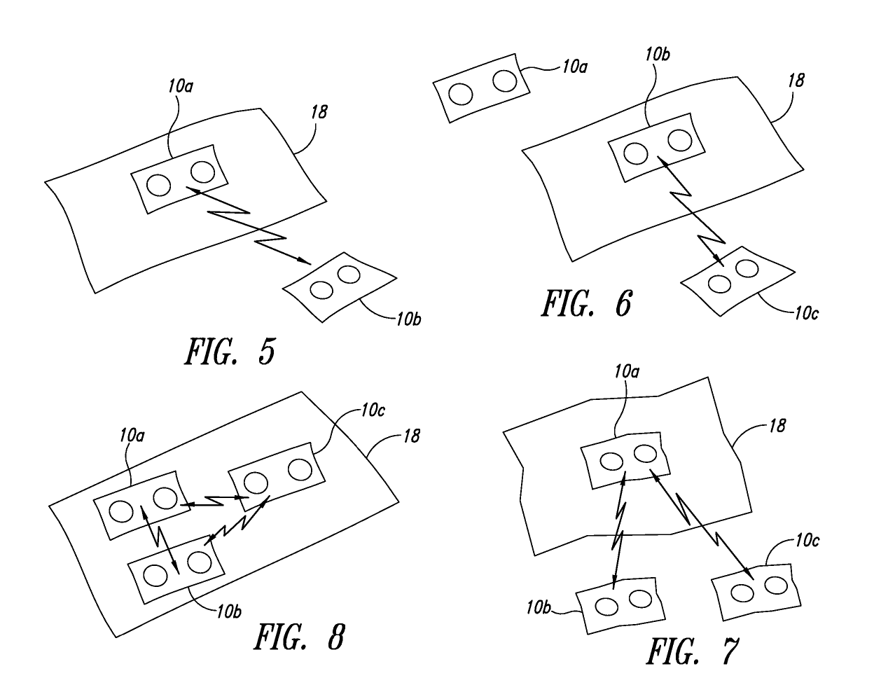

FIG. 7 shows a first active agent delivery device 10a applied to the biological interface 18, and communicating with both a second and third active agent delivery devices 10b, 10c, respectively, which are not applied to the biological interface 18. Additionally, the second and third active agent delivery devices 10b, 10c may wireless communicate with each other.

FIG. 8 shows a first, second, and third active agent delivery devices 10a-10c, respectively, applied to the biological interface 18 at distinct portions thereof, to deliver respective active agents to the biological entity. The first, second, and third active agent delivery devices 10a-10c can wirelessly communicate parameter and other performance information between each other, and adjust active agent delivery accordingly. Where the first, second, and third active agent delivery devices 10a-10c are widely spaced with respect to one another, the first, second, and third active agent delivery devices 10a-10c may act as a repeater system, the second active agent delivery device 10c forwarding information received from the first active agent delivery device 10a to the third active agent delivery device 10c.

The above described embodiments may advantageously employ a greater number of active agent delivery devices 10, and which may delivery active agent simultaneously and/or sequentially.

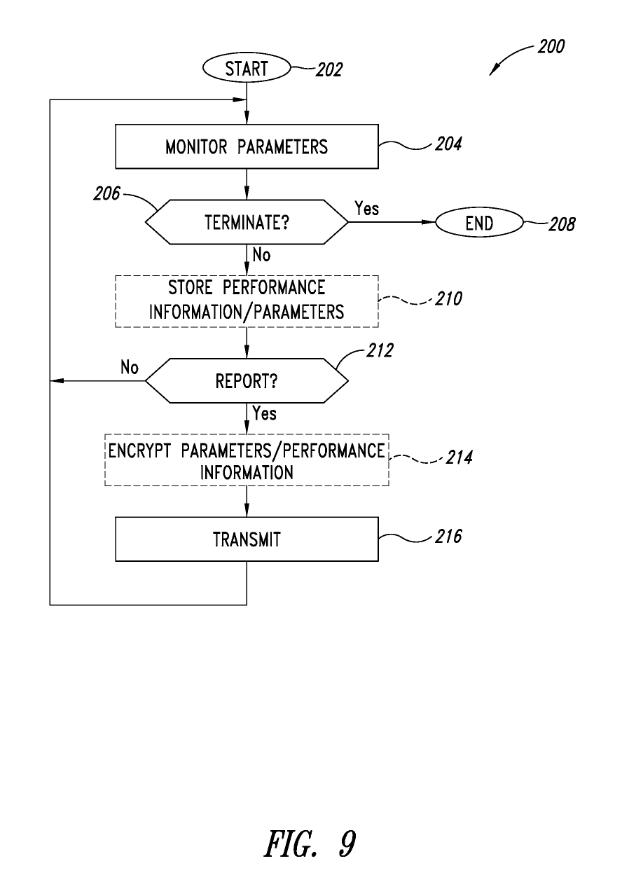

FIG. 9 is a high level flow diagram of a method 200 of operating an active agent delivery device 10 to monitor and report parameters and/or performance information, according to one illustrated embodiment. The method 200 may be implement by the controller 96, as either software or firmware instructions, or as hardwired logic.

The method 200 starts at 202, for example in response to an activation of the active agent delivery device 10. As discussed in more detail below, at 204 the controller 96 monitors the parameters and/or performance of the active agent delivery device 10.

At 206, the controller 96 determines whether or not to terminate the method 200. As discussed in more detail below, termination may be due to: the expiration of a time period, turning OFF of the device 10, exhaustion of active agent and/or power, or detection of degraded performance or malfunction. In particular, the controller 96 may, for example, check a terminate flag which may be set via another process or thread. If the terminate flag is set to a logical value corresponding to yes, the method 200 terminates at 208. Otherwise the method 200 passes control to 210 or 212.

Optionally, at 210, the controller 96 stores parameters and/or other performance information. The storage may be to one or more registers of the controller 96, or memory structures (not shown) associated with the controller 96, such as random access memory (RAM).

At 212, the controller 96 determines whether or not to wirelessly report the parameters and/or other performance information. As discussed in more detail below, reporting may be in response to an inquiry or interrogation, for example, from another active agent delivery device, and/or in response to the expiration of a period or time. In particular, the controller 96 may, for example, check a report flag which may be set via another process or thread. If the report flag is set to a logical value corresponding to yes, the method 200 passes control to 214 or 216. Otherwise the method 200 passes control back to 204.

Optionally at 214, the controller 96 encrypts the parameters and/or other performance information. Encryption advantageously reduces the ability of third parties to mischievously interfere with the provisional of medical services. Encryption also advantageously protects personal medical information, which may be a legal requirement in some jurisdictions. The controller 96 may employ any of a variety of standard encryption algorithms. For example, the controller 96 may employ an encryption algorithm based on public/private key pairs. The public key may belong to a specific active agent delivery device to which the information will be sent, or may be generic to a few or a large number of active agent delivery devices.

At 216, the controller 96 transmits the parameters and/or other performance information. The controller 96 may forward appropriate signals to the transmitter 108a of the transceiver 108 to cause transmission of the parameters and/or other performance information. The active agent delivery device 10 may include additional structures, such as a digital-to-analog converter between the controller 96 and transmitter 18a. Alternatively, the transceiver may implement a digital-to-analog conversion, if necessary or convenient.

The transmission may be a broadcast, or alternatively a pointcast. The transmission can employ any known or later developed protocol, including: time division multiple access (TDMA), frequency division multiple access (FDMA), code division multiple access (CDMA), spread spectrum, and/or BLUETOOTH®.

After transmission, control returns to 204.

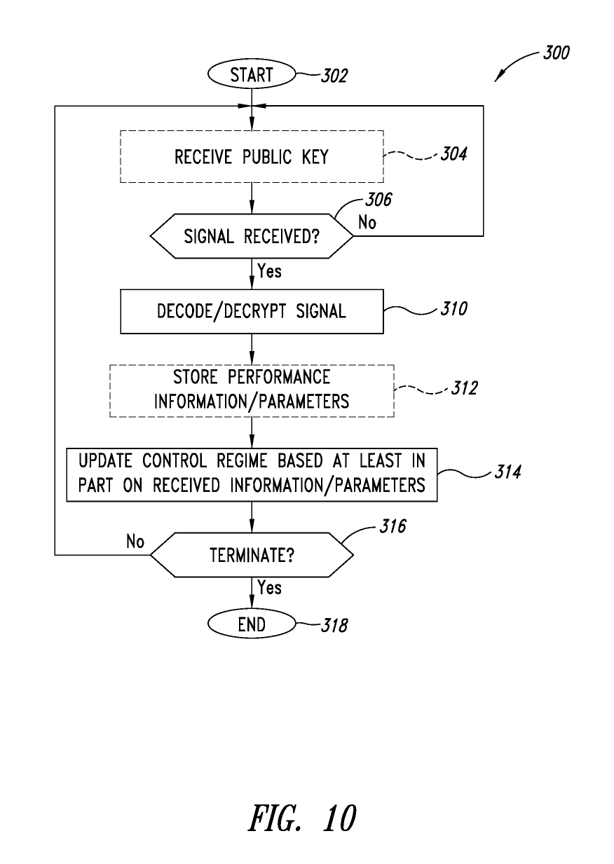

FIG. 10 is a high level flow diagram of a method 300 of operating an active agent delivery device to receive parameters and/or performance information and modify active agent delivery in response thereto, according to one illustrated embodiment. The method 300 may be implement by the controller 96, as either software or firmware instructions, or as hardwired logic.

The method 300 starts at 302, for example in response to an activation of the active agent delivery device 10.

Optionally, at 304 the controller 96 receives a public key form another active agent delivery device 10. This permits the controller to encrypt parameters and other performance information to be sent to the specific other active agent delivery device 10.

At 306, the controller 96 determines whether a signal is received. The controller 96 may use any of a variety of known or later developed methods and circuits for detecting the receipt of a transmission. If a signal is not received, a wait loop is executed, with control passing back to 304. If a signal is received, control passes to 310.

Optionally at 310, the controller 96 decrypts and/or decodes the received signal. For example, the controller may decrypt the signal using use a private key previously provided by the active agent delivery device 10 to other active agent delivery devices, or using a generic private key common to an number of active agent delivery devices. The controller 96 may decode the information using any suitable decoding methods or structures currently know or later developed. Such methods and/or structures are commonly known in the telecommunications industry (TDMA, FDMA, CDMA), and may, for example, include up and/or down mixers.

Optionally at 312, the controller 96 stores parameters and/or other performance information. The storage may be to one or more registers of the controller 96, or memory structures (not shown) associated with the controller 96, such as random access memory (RAM).

At 316, the controller 96 determines whether or not to terminate the method 300. As discussed in more detail below, termination may be due to: the expiration of a time period, turning OFF of the device 10, exhaustion of active agent and/or power, or detection of degraded performance or malfunction. In particular, the controller 96 may, for example, check a terminate flag which may be set via another process or thread. If the terminate flag is set to a logical value corresponding to yes, the method 300 terminates at 318. Otherwise the method 300 passes control to 304 and waits for receipt of further signals.

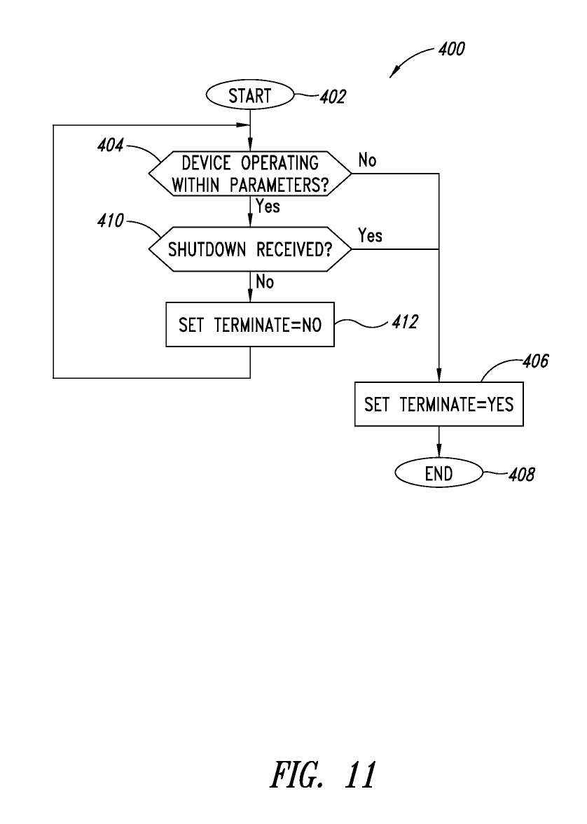

FIG. 11 is a low level flow diagram of a method 400 of determining whether to terminate operation according to one illustrated embodiment, the method useful in the methods of FIGS. 9 and 10.

The method 400 starts at 402. For example, the method 400 may start in response to an activation of the active agent deliver device 10, and may run in parallel with the methods 200 and/or 300, for example as a separate process or thread. Activation may be the closing of a switch, or simply the application of the active agent delivery device 10 to the biological interface 18 that completes the circuit. Alternatively, the method 400 may start in response to a call from the controller 96, for example, at 206 of method 200 (FIG. 9) and/or 316 of method 300 (FIG. 10).

At 404, the controller 96 determines whether the active agent delivery device 10 is operating within defined parameters. The controller may compare one or more of the monitored parameters with one or more respective thresholds. If the active agent delivery device 10 is not operating within defined parameters, control passes to 406 where a termination flag is set (e.g., YES) and the method 400 terminates at 408. Otherwise control passes to 410.

At 412, the controller 96 determines whether a shut down command has been received. The shut down command may be generated by the opening of a switch, or simply the removal of the active agent delivery device 10 from the biological interface, opening the circuit between the electrode elements 24, 68. Additionally, or alternatively, the shut down command can be generated by another active agent delivery device or by some other external controller. If a shut down command has been received, control passes to 406 where the terminate flag is set (e.g., YES), and the process or thread implementing method 400 terminates at 408. If a shut down command has not been received, control returns to 404, where the process or thread implementing the method 400 continues.

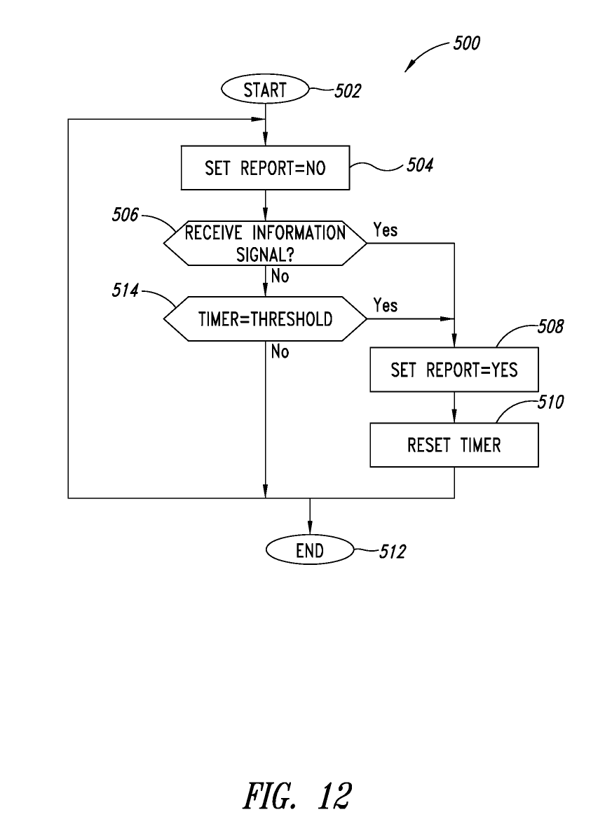

FIG. 12 is a low level flow diagram of a method 500 of determining whether to report parameters and/or performance information according to one illustrated embodiment, the method useful in the method of FIG. 9. The method 500 may be implement by the controller 96, as either software or firmware instructions, or as hardwired logic.

The method 500 starts at 502. For example, the method 500 may start in response to an activation of the active agent deliver device 10, and may run in parallel with the methods 200 and/or 300, for example as a separate process or thread. Activation may be the closing of a switch, or simply the application of the active agent delivery device 10 to the biological interface 18 that completes the circuit. Alternatively, the method 500 may start in response to a call from the controller 96, for example, at 212 of method 200 (FIG. 9).

At 504, the controller 96 sets a report flag to an appropriate logical value (e.g., NO). Optionally at 506, the controller 96 determines whether an inquiry or interrogation signal has been received. The controller 96 may employ currently known techniques and structures to determine whether an interrogation signal has been reached, for example those employed in radio frequency identification (RFID).

If an interrogation signal has been received, the controller 96 sets the report flag to an appropriate logical value (e.g., YES) at 508 and resets a timer or clock at 510. The controller 96 then optionally terminates the method 500 at 512 (broken line arrow), or returns control to 504 (solid line arrow).

If an interrogation signal has not been received, the controller 96 determines whether the timer or clock as reached a reporting threshold at 514. The reporting threshold may be preconfigured, or may be user configurable, or automatically configurable based on an active agent delivery regime. If the timer or clock has reached the reporting threshold, the controller 96 sets the report flag to an appropriate logical value (e.g., YES) at 508 and resets a timer or clock at 510. The controller 96 then optionally terminates the method 500 at 512 (broken line arrow), or returns control to 504.

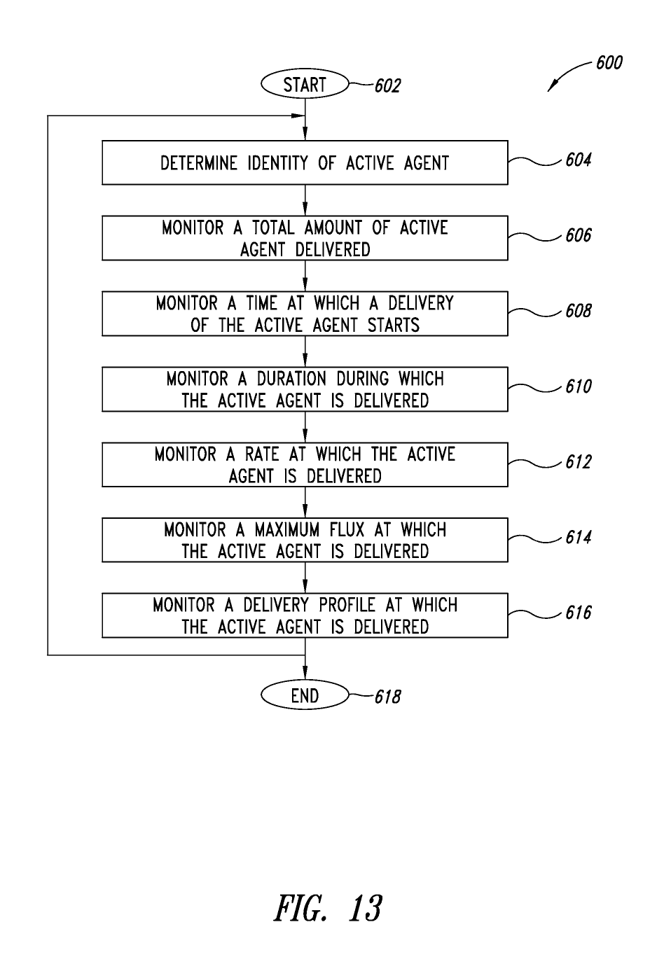

FIG. 13 is a low level flow diagram of a method 600 of monitoring parameters and/or performance information according to one illustrated embodiment, the method useful in the method of FIG. 9. The method 600 may be implement by the controller 96, as either software or firmware instructions, or as hardwired logic.

The method 600 starts at 602. For example, the method 600 may start in response to an activation of the active agent deliver device 10, and may run in parallel with the methods 200 and/or 300, for example as a separate process or thread. Activation may be the closing of a switch, or simply the application of the active agent delivery device 10 to the biological interface 18 that completes the circuit. Alternatively, the method 600 may start in response to a call from the controller 96, for example, at 204 of method 200 (FIG. 9).

At 604, the controller 96 monitors an identity of the active agent. The controller 96 may monitor an identifier that identifies the type of active agent (e.g., lidocaine chloride, 0.3%), or that unique identifies the unit or batch of active agent, for example via a unique serial number. Such may be encoded in the active agent reservoir, or active agent delivery device 10, for example hardwired in control circuitry, or as an RFID transponder, or using an electronic article surveillance type tag. The controller 96 may be able to read such identifier using the antenna 110a, 110b, and transceiver 108, or by using a separate antenna and receiver (not shown).

At 606, the controller 96 monitors a total amount of active agent delivered. For example, the controller 96 may monitor a current through a reservoir, membrane or other structure, and/or may monitor a voltage across a reservoir, membrane or other structure to determine the total amount of active agent delivered. For instance, the controller 96 may monitor the amount of current drawn over an entire period of time during which active agent is delivered, and determine the amount of active agent delivery based on a defined relationship current and rate of active agent delivery, based on the knowledge of the total time of delivery. Such may be refined using empirically derived relationships.

At 608, the controller 96 monitors a time at which a delivery of the active agent starts. For example, the controller 96 may start a timer or clock when current beings to flow, for example in response to activation of a switch or simply the completion of the circuit by the placement of the active agent delivery device 10 on the biological interface 18 (FIG. 1).

At 610, the controller 96 monitors a duration during which the active agent is delivered. For example, the controller 96 may stop a timer or clock when current stops flowing, for example in response to deactivation of a switch or simply the opening of the circuit path between the electrode assemblies 12, 14 by the removal of the active agent delivery device 10 from the biological interface 18 (FIG. 1).

At 612, the controller 96 monitors a rate at which the active agent is delivered. For example, the controller 96 may monitor a current through a reservoir, membrane or other structure, and/or may monitor a voltage across a reservoir, membrane or other structure to determine the rate at which the active agent is delivered. For instance, the controller 96 may monitor an instantaneous rate based on a relationship between current and rate of delivery and a knowledge of the instantaneous current. Also for instance, the controller 96 may monitor an average rate by cumulating or integrated the instantaneous rates.

At 614, the controller 96 monitors a maximum flux at which the active agent is delivered. For example, the controller 96 may monitor a current through a reservoir, membrane or other structure, and/or may monitor a voltage across a reservoir, membrane or other structure to determine the maximum flux at which the active agent is delivered. For instance, the controller 96 may monitor the maximum current draw. The controller 96 may determine the maximum flux based on a relationship between current and rate of delivery, and a knowledge of the maximum current draw.

At 616, the controller 96 monitors a delivery profile at which the active agent is delivered. For example, the controller 96 may monitor a current through a reservoir, membrane or other structure, and/or may monitor a voltage across a reservoir, membrane or other structure to determine the total amount of active agent delivered. For instance, the controller 96 may monitor the current over time, determining the delivery profile based at least in part on a relationship between current and rate of delivery, and a knowledge of the instantaneous current through the active agent delivery. Such may be refined using empirically derived relationships, for example, a relationship between rate of delivery and voltage, a relationship between rate of delivery and impedance where impedance is either monitored or determined from another monitored parameter (e.g., current or voltage).

The controller 96 may terminate the method 600 at 618 (broken line arrow), or may return control to 604.

The controller 96 may execute the method 600 omitting some of the acts and/or adding additional acts. Additionally, or alternatively, the controller 96 may execute the method 600 in a different order, or may execute with a difference frequency of some acts with respect to other acts. For example, the controller 96 may monitor the identity of the active agent only once at startup, while monitoring a rate of delivery more frequently, for example once ever half second.

FIG. 14 is a low level flow diagram of a method 700 of monitoring parameters and/or performance by monitoring a current through a reservoir, membrane or other structure of the active agent delivery device according to one illustrated embodiment, the method useful in the method of FIG. 9.

At 702, the controller 96 monitors the current through at least one reservoir, membrane or other structure of the active agent delivery device 10. The controller 96 may rely on signals i₁-iₙ (FIG. 1), indicative of current sensed or measured by current sensors 102a-102d or other current sensors (not shown).

As suggested above, the current may be a useful parameter in and of itself, and may also be used to derive other useful parameters and/or other performance information. Such may be useful in monitoring active agent delivery. Such may also be useful in monitoring other performance information. For example, a low value of current can be indicative of, for example, increased impedance that may be caused by poor conduction between and/or improper placement of one or both of the electrode assemblies 12 and 14 on the biological interface 18. The poor conduction can be caused, for instance, if residue from the outer release liner 46 is still present and inhibiting ionic flow. Increased impedance may also be indicative of a loose conductive connection between the power supply 16 and one (or both) of the electrode assemblies 12 and 14. Increased impedance may also be further indicative of poor ionic flow or charge transfer through the various membranes of the active electronic assembly 12, which may be due to a number of abnormal factors, such as neutralized ions, faulty membranes, low active agent concentration, and others. A high detected current value can be indicative of a short circuit somewhere in the iontophoresis device 10.

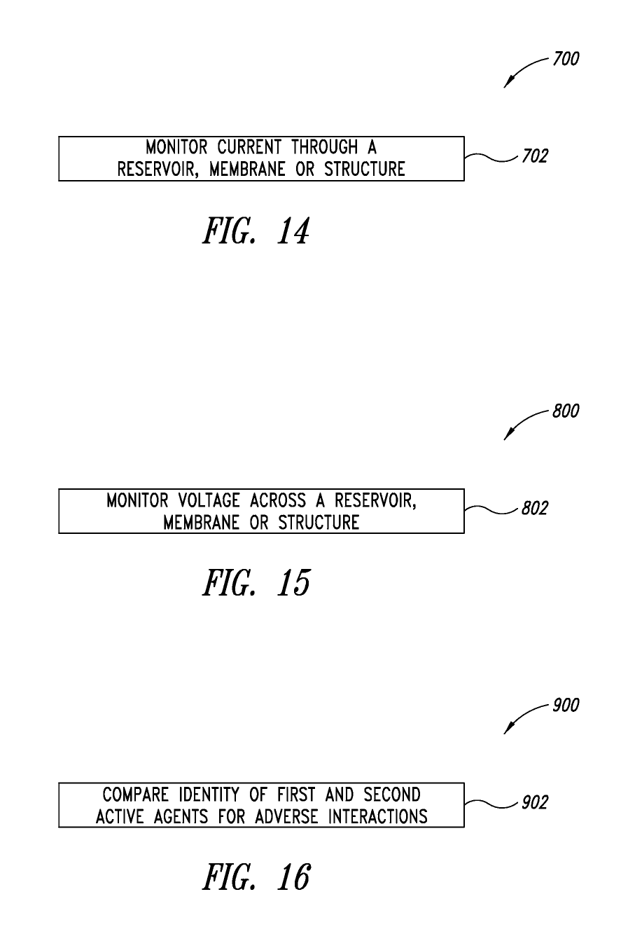

FIG. 15 is a low level flow diagram of a method 800 of monitoring parameters and/or performance by monitoring a voltage across a reservoir, membrane or other structure of the active agent delivery device according to one illustrated embodiment, the method useful in the method of FIG. 9.

At 802, the controller 96 monitors the voltage across at least one reservoir, membrane or other structure of the active agent delivery device 10. The controller 96 may rely on signals v₁-vₘ (FIG. 1), indicative of voltage sensed or measured by voltage sensors 104a-104c, or other voltage sensors (not shown).

As suggested above, the current may be a useful parameter in and of itself, and may also be used to derive other useful parameters and/or other performance information. Such may be useful in monitoring active agent delivery. Such may also be useful in monitoring other performance information. For example, a high or increase in detected voltage value across the active electrode assembly 12 can be indicative of, for example, increased impedance. As discussed above, increased impedance can be indicative of improper electrode placement, a defect, or other malfunction. Conversely, a low detected voltage can be indicative of a short circuit somewhere in the iontophoresis device 10.

FIG. 16 is a low level flow diagram of a method 900 of monitoring parameters and/or performance information by comparing an identity of first and second active agents for adverse interactions, according to one illustrated embodiment, the method useful in the method of FIG. 9.

At 902, the controller 96 compares an identity of an active agent to be delivered by the first active agent delivery device 10a (FIGS. 5-8) with an identity of an active agent previously delivered, currently being delivered or that will be delivered by a second active agent delivery device 10b. The controller 96 may optionally rely on a lookup table or algorithm for converting an identifier, for example a serial number, into another identifier that identifies the active agent.

The controller 96 may use a look up table to determine whether the combination of two or more active agents has been identified as being either acceptable or unacceptable, due to potential or likely adverse interactions between the active agents. In one embodiment, the controller(s) 96 of one or more active agent devices 10a-10c automatically prevent delivery of the active agent, and/or presents a human-perceptible indication if the combination has been identified as presenting potential adverse interactions. In another embodiment, the controller(s) 96 of one or more active agent devices 10a-10c automatically prevent delivery of the active agent, and/or presents a human-perceptible indication if the combination has not been identified as safe from adverse interactions. This embodiment provides a fail safe type mechanism.

Additionally, or alternatively, the controller 96 may employ a look up table that lists active agents that are not to be delivered to the particular patient or subject. Such may include active agents to which the patient or subject has a known adverse reaction, and/or active agents for which it is not known whether the patient or subject may have an adverse reaction.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular active agents may thus be carried through the biological interface into the underlying tissues primarily via electroosmotic solvent flow. In certain embodiments, the high molecular weight polyelectrolytic active agents may be proteins, polypeptides or nucleic acids.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiment may include a control circuit or subsystem to control a voltage, current or power applied to the active and counter electrode elements 20, 40. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having an active agent solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; an electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488,970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; Japanese patent application Serial No. 2005-081220, filed Mar. 22, 2005; and U.S. Provisional Patent Application No. 60/722,088, filed Sep. 30, 2005.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

Some embodiments may advantageously employ existing communications protocols and standards, for example BLUETOOTH®.

While the illustrated embodiments show an antenna 110 and transceiver 108 for wirelessly communicating using radio signals (e.g., signals in the radio, microwave or other portions of the electromagnetic spectrum), other embodiments may use other components to provide wireless communications. For example, some embodiments may employ a light source (e.g., LED) and light detector (e.g., photodiode or photodetector) to provide wireless communications. Such may communicate in visible or non-visible portions of the electromagnetic spectrum, for example the infrared portion.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An active agent delivery device operable to deliver an active agent to a biological entity, the device comprising:
an active agent reservoir to hold a quantity of the active agent;
a power source operable to supply power to actively transfer at least some of the active agent from the active agent delivery device to the biological interface;
a monitoring circuit operable to monitor at least one parameter indicative of a transfer of active agent from the device;
at least a first antenna; and
a transmitter coupled to the at least one antenna to transmit a signal indicative of at least one of the monitored parameters.

2. The active agent delivery device of claim 1 wherein the active agent delivery device is an iontophoresis device comprising an active electrode assembly and a counter electrode assembly, the active electrode assembly including an active electrode element operable to apply an electrical potential from the power source to transfer at least some of the active agent from the active electrode assembly, and the counter electrode assembly including a counter electrode element to provide a return current path from the biological entity to the power source.

3. The active agent delivery device of claim 2 wherein the first antenna is positioned proximate and overlying one of the active or counter electrode elements, the antenna and active or counter electrode element forming a directional antenna system.

4. The active agent delivery device of claim 3 wherein the first antenna is spaced distally from the active electrode element with respect a biological interface contacting portion of the active electrode assembly when in use.

5. The active agent delivery device of claim 2 wherein the active electrode assembly further includes an electrolyte reservoir proximate the active electrode, an outermost selective membrane position proximate an exterior of the active agent delivery device; and an inner ion selective membrane positioned between the electrolyte reservoir and the outermost ion exchange membrane.

6. The active agent delivery device of claim 5 wherein the outermost ion selective membrane is a first ion exchange membrane substantially passing ions having a first polarity the same as a polarity of the active agent and substantially blocking passage of ions having a second polarity opposite the first polarity, and wherein the inner ion selective membrane is a second ion exchange membrane substantially passing ions having the second polarity and substantially blocking ions having the first polarity.

7. The active agent delivery device of claim 1, further comprising:
a receiver coupled to the at least one antenna to receive a signal indicative of at least one parameter indicative of a transfer of active agent from a different active agent delivery device.

8. The active agent delivery device of claim 7 wherein there is only a single antenna and the transmitter and the receiver are formed as a transceiver both communicatively coupled to the single antenna.

9. The active agent delivery device of claim 1 wherein the monitoring circuit monitors a total amount of active agent delivered by the active agent delivery device.

10. The active agent delivery device of claim 1 wherein the monitoring circuit monitors a time at which a delivery of the active agent by the active agent delivery device starts.

11. The active agent delivery device of claim 1 wherein the monitoring circuit monitors a duration during which the active agent is delivered by the active agent delivery device.

12. The active agent delivery device of claim 1 wherein the monitoring circuit monitors a rate at which the active agent is delivered by the active agent delivery device.

13. The active agent delivery device of claim 1 wherein the monitoring circuit monitors a maximum flux at which the active agent is delivered by the active agent delivery device.

14. The active agent delivery device of claim 1 wherein the monitoring circuit monitors a delivery profile at which the active agent is delivered by the active agent delivery device.

15. An active agent delivery system operable to control delivery of an active agent to a biological entity, the system comprising:
a first active agent delivery device including an active agent reservoir to hold a quantity of the active agent, a control circuit operable to control and monitor at least one aspect of a delivery of the active agent from the first active agent delivery device, at least one antenna operable to transmit a signal indicative of at least one of the monitored aspects; and
at least a second active agent delivery device including an active agent reservoir to hold a quantity of the active agent, a control circuit operable to control and monitor at least one aspect of a delivery of the active agent from the second active agent delivery device, at least one antenna operable to receive the signal indicative of at least one of the monitored aspects of the first active agent delivery device.

16. The active agent delivery system of claim 15 wherein the control circuit of the second active agent delivery device is responsive to the received signal indicative of at least one of the monitored aspects of the first active agent delivery device.

17. The active agent delivery system of claim 15 wherein the control circuit of the second active agent delivery device is operable to modify at least one aspect of the delivery of the active agent from the second active agent delivery device based at least in part on the received signal indicative of at least one of the monitored aspects of the first active agent delivery device.

18. The active agent delivery system of claim 15 wherein the antenna of the second active agent delivery device is further operable to transmit a signal indicative of at least one of the monitored aspects of the delivery of the active agent from the second active agent delivery device, and further comprising:
at least a third active agent delivery device including an active agent reservoir to hold a quantity of the active agent, a control circuit operable to control and monitor at least one aspect of a delivery of the active agent from the third active agent delivery device, at least one antenna operable to receive the signals indicative of at least one of the monitored aspects of the first and the second active agent delivery devices.

19. The active agent delivery system of claim 18 wherein the control circuit of the third active agent delivery device is responsive to the received signal indicative of at least one of the monitored aspects of the first and the second active agent delivery devices.

20. The active agent delivery system of claim 15 wherein the control circuits of the first and the second active agent delivery devices monitor a total amount of active agent delivered by the respective active agent delivery devices.

21. The active agent delivery system of claim 15 wherein the control circuits of the first and the second active agent delivery devices monitor a time at which a delivery of the active agent by the respective active agent delivery device starts.

22. The active agent delivery system of claim 15 wherein the control circuits of the first and the second active agent delivery devices monitor a duration during which the active agent is delivered by the respective active agent delivery device.

23. The active agent delivery system of claim 15 wherein the control circuits of the first and the second active agent delivery devices monitor a rate at which the active agent is delivered by the respective active agent delivery device.

24. The active agent delivery system of claim 15 wherein the control circuits of the first and the second active agent delivery devices monitor a maximum flux at which the active agent is delivered by the respective active agent delivery device.

25. The active agent delivery system of claim 15 wherein the control circuits of the first and the second active agent delivery devices monitor a delivery profile at which the active agent is delivered by the respective active agent delivery device.

26. The active agent delivery system of claim 15 wherein the first and the second active agent delivery devices are respective an iontophoresis devices, each comprising an active electrode assembly and a counter electrode assembly, the active electrode assembly including an active electrode element operable to apply an electrical potential from the power source to transfer at least some of the active agent from the active electrode assembly, and the counter electrode assembly including a counter electrode element to provide a return current path from the biological entity to the power source.

27. A method of operating at least a first and a second active agent delivery device to deliver an active agent to a biological entity, the method comprising:
delivering a quantity of an active agent from the first active agent delivery device;
monitoring at least one aspect of the delivery of the active agent from the first active agent delivery device;
transmitting a signal to the second active agent delivery device at least indicative of the at least one monitored aspect of the delivery of the active agent from the first active agent delivery device; and
delivering a quantity of an active agent from the second active agent delivery device, based in part on information in the signal received from the first active agent delivery device.

28. The method of claim 27 wherein monitoring at least one aspect of the delivery of the active agent from the first active agent delivery device comprises measuring at least one parameter indicative of a total amount of active agent delivered by the respective active agent delivery devices.

29. The method of claim 27 wherein monitoring at least one aspect of the delivery of the active agent from the first active agent delivery device comprises measuring at least one parameter indicative of a time at which a delivery of the active agent by the respective active agent delivery device starts.

30. The method of claim 27 wherein monitoring at least one aspect of the delivery of the active agent from the first active agent delivery device comprises measuring at least one parameter indicative of a duration during which the active agent is delivered by the respective active agent delivery device.

31. The method of claim 27 wherein monitoring at least one aspect of the delivery of the active agent from the first active agent delivery device comprises measuring at least one parameter indicative of a rate at which the active agent is delivered by the respective active agent delivery device.

32. The method of claim 27 wherein monitoring at least one aspect of the delivery of the active agent from the first active agent delivery device comprises measuring at least one parameter indicative of a maximum flux at which the active agent is delivered by the respective active agent delivery device.

33. The method of claim 27 wherein monitoring at least one aspect of the delivery of the active agent from the first active agent delivery device comprises measuring at least one parameter indicative of a delivery profile at which the active agent is delivered by the respective active agent delivery device.

34. The method of claim 27 wherein monitoring at least one aspect of the delivery of the active agent from the first active agent delivery device comprises measuring at least one parameter indicative of an functioning/malfunctioning operational status of the first active agent delivery device.

35. The method of claim 27 wherein monitoring at least one aspect of the delivery of the active agent from the first active agent delivery device comprises determining an identity of the first active agent.

36. The method of claim 27, further comprising:
determining to deliver the second active agent based at least in part on the identify of the first active agent and an identity of the second active agent.

37. The method of claim 36 wherein determining to deliver the second active agent based at least in part on the identify of the first active agent and an identity of the second active agent comprises determining whether a combination of the first and the second active agents is identified as presenting an adverse reaction problem.

38. The method of claim 27 wherein monitoring at least one aspect of the delivery of the active agent from the first active agent delivery device comprises measuring a current flow through at least one portion of the first active agent delivery device.

39. The method of claim 27 wherein monitoring at least one aspect of the delivery of the active agent from the first active agent delivery device comprises measuring a voltage across at least one portion of the first active agent delivery device.

40. A method of operating a plurality of active agent delivery devices to deliver active agents to a biological entity, the method comprising:
delivering a quantity of a first active agent from a first one of the active agent delivery devices;
monitoring at least one aspect of the delivery of the first active agent from the first active agent delivery device;
transmitting a signal to at least a second one of the active agent delivery devices indicative of the at least one monitored aspect of the delivery of the first active agent from the first active agent delivery device;
delivering a quantity of a second active agent from the second active agent delivery device, based in part the at least one monitored aspect of delivery of the first active agent from the first active agent delivery device;
monitoring at least one aspect of the delivery of the second active agent from the second active agent delivery device;
transmitting a signal to at least a third one of the active agent delivery devices indicative of the at least one monitored aspect of the delivery of the second active agent from the second active agent delivery device; and
delivering a quantity of a third active agent from the third active agent delivery device, based in part on the at least one monitored aspect of delivery of at least one of the first and the second active agents from the first and the second active agent delivery devices.

41. The method of claim 40 wherein delivering a quantity of a first active agent from a first one of the active agent delivery devices includes delivering a first quantity of a compound, and wherein delivering a quantity of a second active agent from a second one of the active agent delivery devices includes delivering a second quantity of the compound.

42. The method of claim 41 wherein the first quantity and the second quantities are equal.

43. The method of claim 40 wherein monitoring at least one aspect of the delivery comprises measuring at least one parameter indicative of a total amount of the first and the second active agents delivered by the first and the second active agent delivery devices, respectively.

44. The method of claim 40 wherein monitoring at least one aspect of the delivery comprises measuring at least one parameter indicative of a duration during which the first and the second active agents are delivered by the first and the second active agent delivery devices, respectively.

45. The method of claim 40 wherein monitoring at least one aspect of the delivery comprises measuring at least one parameter indicative of a rate at which the first and the second active agents are delivered by the first and the second active agent delivery devices, respectively.

46. The method of claim 40 wherein monitoring at least one aspect of the delivery comprises measuring at least one parameter indicative of a maximum flux at which the first and the second active agents are delivered by the first and the second active agent delivery devices, respectively.

47. The method of claim 40 wherein monitoring at least one aspect of the delivery comprises measuring at least one parameter indicative of a delivery profile at which the first and the second active agents are delivered by the first and the second active agent delivery devices, respectively.

48. The method of claim 40 wherein monitoring at least one aspect of the delivery comprises determining an identity of the first and the second active agents delivered by the first and the second active agent delivery devices, respectively.

49. The method of claim 40, further comprising:
receiving an interrogation signal at the fist active agent delivery device from the second active agent delivery device, wherein transmitting a signal to at least a third one of the active agent delivery devices indicative of the at least one monitored aspect of the delivery of the second active agent from the second active agent delivery device is responsive to receiving the interrogation signal from the second active agent delivery device.

50. The method of claim 40, further comprising:
encrypting each of the signals before transmitting the signals.

51. The method of claim 40, further comprising:
receiving a public key from the second active agent delivery;
encrypting the signal using the public key before transmitting the signal to at least the second active agent delivery devices.
